(19) United States
(12) Patent Application Publication
(10) Pub. No.: US 2010/0278824 A1
(43) Pub. Date: Nov. 4, 2010

(54) COMBINATIONS COMPRISING GEMCITABINE AND TYROSINE KINASE INHIBITORS FOR THE TREATMENT OF PANCREATIC CANCER

(76) Inventor: Isaiah Josh Fidler, Houston, TX (US)

Correspondence Address: NOVARTIS;CORPORATE INTELLECTUAL PROPERTY, ONE HEALTH PLAZA 101/2, EAST HANOVER, NJ, 07936-1080, US

(21) Appl. No.: 12/834,676

(22) Filed: Jul. 12, 2010

Related U.S. Application Data
(63) Continuation of application No. 11/995,388, filed on Jan. 11, 2008.
(60) Provisional application No. 60/702,963, filed on Jul. 27, 2005.

(51) Int. Cl. A61K 39/395; A61K 31/502; A61K 31/5377; A61K 31/519; A61K 31/7068; A61P 35/00

(52) U.S. Cl. 424/133.1; 514/248; 514/234.5; 514/265.1; 514/49

(57) ABSTRACT

The invention relates to a method of treating a warm-blooded animal having pancreatic cancer, in particular it relates
to a method comprising administering to the warm-blooded animal having pancreatic cancer a dual inhibitor of the epidermal growth factor receptor (EGF-R) tyrosine kinase activity and the vascular endothelial growth factor receptor (VEGF-R) tyrosine kinase activity,
to a method comprising administering to the warm-blooded animal having pancreatic cancer a combination comprising (a) a compound which decrease the activity of the EGF and (b) a compound which decreases the activity of VEGF,
to a combination comprising (a) a dual inhibitor of the EGF-R tyrosine kinase activity and the VEGF-R tyrosine kinase activity or, alternatively, a compound which decrease the activity of the EGF and a compound which decreases the activity of VEGF and (b) at least one compound selected from an inhibitor of the platelet derived growth factor receptor (PDGF-R) tyrosine kinase activity and antineoplastic anti-metabolites;
to a method of treating a warm-blooded animal having pancreatic cancer comprising administering to the warm-blooded animal said combination;
to the use of such a combination for the preparation of a medicament for the treatment of pancreatic cancer; and
to a commercial package or product comprising such a combination together with instructions for the treatment of pancreatic cancer.

The invention relates to a method of treating a warm-blooded animal having pancreatic cancer, in particular it relates
to a method comprising' administering to the warm-blooded animal having pancreatic cancer a dual inhibitor of the epidermal growth factor receptor (EGF-R) tyrosine kinase activity and the vascular endothelial growth factor receptor (VEGF-R) tyrosine kinase activity,
to a method of treating a warm-blooded animal having pancreatic cancer comprising administering to the warm-blooded animal a combination comprising (a) a compound which decrease the activity of the EGF and (b) a compound which decreases the activity of VEGF,
to a combination comprising (a) a dual inhibitor of the EGF-R tyrosine kinase activity and the VEGF-R tyrosine kinase activity or, alternatively, a compound which decrease the activity of the EGF and a compound which decreases the activity of VEGF and (b) at least one compound selected from an inhibit& of the platelet derived growth factor receptor (PDGF-R) tyrosine kinase activity and antineoplastic anti-metabolites;
to a method of treating a warm-blooded animal having pancreatic cancer comprising administering to the warm-blooded animal said combination;
to the use of such a combination for the preparation of a medicament for the treatment of pancreatic cancer; and
to a commercial package or product comprising such a combination together with instructions for the treatment of pancreatic cancer.

Pancreatic adenocarcinoma is one of the most aggressive malignancies with most patients developing metastatic disease. Although gemcitabine can prolong survival of patients, only less than 3% survive 5 years after the initial diagnosis and the median survival duration is les than 6 months. Clearly, there is an urgent need to develop new treatment options for pancreatic cancer.

Surprisingly, in a first aspect of the present invention it was found that simultaneous inhibition of EGFR and VEGFR inhibits the growth of human pancreatic carcinoma. In a further aspect, it was found that simultaneous inhibition of EGFR, VEGFR and, optionally, PDGFR, in combination with administration of an antineoplastic anti-metabolite inhibits very strongly the growth of human pancreatic carcinoma and represents a perspective for prolongation of survival compared to administration of the antineoplastic anti-metabolite alone.

Hence, the present invention relates to the use of a dual inhibitor of the EGF-R tyrosine kihase activity and the VEGF-R tyrosine kinase activity or, alternatively, the use of a combination comprising (a) a compound which decrease the activity of EGF and (b) a compound which decreases the activity of VEGF for the preparation of a medicament for the treatment of pancreatic cancer.

Additionally, the present invention relates to a combination comprising (a) a dual inhibitor of the EGF-R tyrosine kinase activity and the VEGF-R tyrosine kinase activity or, alternatively, (a) a compound which decrease the activity of EGF and (b) a compound which decreases the activity of VEGF and (b) at least one compound selected from an inhibitor of the PDGF-R tyrosine kinase activity and antineoplastic anti-metabolites, wherein the active ingredients are present in each case in free form or in the form of a pharmaceutically acceptable salt or any hydrate thereof, and optionally at least one pharmaceutically acceptable carrier; for simultaneous, separate or sequential use in the treatment of pancreatic cancer.

In another aspect, the present invention pertains to a method of treating a warm-blooded animal having pancreatic cancer comprising administering to the warm-blooded animal a dual inhibitor of the EGF-R tyrosine kinase activity and the VEGF-R tyrosine kinase activity in a quantity which is therapeutically effective against pancreatic cancer in which the dual inhibitor of the EGF-R tyrosine kinase activity and the VEGF-R tyrosine kinase activity can also be present in the form of a pharmaceutically acceptable salt as well as to a method of treating a warm-blooded animal having pancreatic cancer comprising administering to the warm-blooded animal a combination comprising (a) a compound which decrease the activity of EGF and (b) a compound which decreases the activity of VEGF in a quantity which is therapeutically effective against pancreatic cancer in which the components of the combination can also be present in the form of their pharmaceutically acceptable salts.

“Dual inhibitor of the EGF-R tyrosine kinase activity and the VEGF-R tyrosine kinase activity” suitable for the present invention are in particular those are described in WO 03/013541, which publication is incorporated by reference, and are 7H-pyrrolo[2,3-d]pyrimidine derivatives of formula (I)

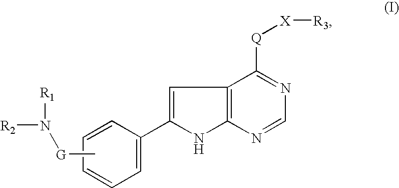

wherein
R₁ and R₂ are each independently of the other hydrogen, unsubstituted or substituted alkyl or cycloalkyl, a heterocyclic radical bonded via a ring carbon atom, or a radical of the formula R₄—Y—(C=Z)- wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen; or
R₁ and R₂ together with the nitrogen atom to which they are attached form a heterocyclic radical;
R₃ is a heterocyclic radical or an unsubstituted or substituted aromatic radical;
G is C₁-C₇-alkylene, —C(═O)—, or C₁-C₆-alkylene-C(═O)— wherein the carbonyl group is attached to the NR₁R₂ moiety;
Q is —NH or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
as well as the salts of these compounds,
   wherein the radicals and symbols have the meanings as defined in WO 03/013541.

In one embodiment, a particularly preferred dual EGF and VEGF protein tyrosine kinase inhibitor for use in the invention is {6-[4-(4-ethyl-piperazin-1-ylmethyl)-phenyl]-7H-pyrrolo[2,3-d]pyrimidin-4-yl}-(1-[phenyl-ethyl)-amine or a pharmaceutically acceptable salt thereof.

N-phenyl-2-pyrimidine-amine derivatives as used herein are those disclosed in U.S. Pat. No. 5,521,184 and US 2004-0102453. N-{5-[4-(4-methyl-piperazino-methyl)-benzoylamido]-2-methylphenyl}-4-(3-pyridyl)-2-pyrimidine-amine can be prepared as disclosed in U.S. Pat. No. 5,521,184 which publication is included into the present patent filing by reference. The monomesylate salt of N-{5-[4-(4-methyl-piperazino-methyl)-benzoylamido]-2-methylphenyl}-4-(3-pyridyl)-2-pyrimidine-amine can be prepared and formulated, e.g., as described in Examples 4 and 6 of WO 99/03854 or as described in WO03/090720, which publications are also included into the present patent filing by reference. The mesylate salt of N-{5-[4-(4-methyl-piperazino-methyl)-benzoylamido]-2-methylphenyl}-4-(3-pyridyl)-2-pyrimidine-amine (Imatinib mesylate, STI571) is marketed under the brand Glivec® (Gleevec®). Glivec® is a tyrosine kinase inhibitor suitable for the treatment of chronic myeloid leukemia and GIST (gastro-intestinal stromal tumors).

The term “antineoplastic anti-metabolites” includes, but is not limited to 5-fluorouracil, capecitabine, gemcitabine, methotrexate and edatrexate. Capecitabine can be administered, e.g., in the form as it is marketed, e.g. under the trademark XELODA™. Gemcitabine can be administered, e.g., in the form as it is marketed, e.g. under the trademark GEMZART™.

Compounds which decreases the activity of VEGF are especially compounds which inhibit the VEGF receptor tyrosine kinase activity, compounds which bind to a VEGF receptor and compounds binding to VEGF, and are in particular the compounds, proteins and monoclonal antibodies generically and specifically disclosed in WO 98/35958, WO 00/09495, WO 00/27820, WO 00/59509, WO 98/11223, WO 00/27819 and EP 0 769947; those as described by M. Prewett et al in Cancer Research 59 (1999) 5209-5218, by F. Yuan et al in Proc. Natl. Acad. Sci. USA, vol. 93, pp. 14765-14770, December 1996, by Z. Zhu et at in Cancer Res. 58, 1998, 3209-3214, and by J. Mordenti et at in Toxicologic Pathology, Vol. 27, no. 1, pp 14-21, 1999; in WO 00/37502 and WO 94/10202; Angiostatin™, described by M. S. O'Reilly et al, Cell 79, 1994, 315-328; and Endostatin™, described by M. S. O'Reilly et al, Cell 88, 1997, 277-285;

compounds which decrease the activity of EGF are especially compounds which inhibit the EGF receptor tyrosine kinase activity, compounds which bind to the EGF receptor and compounds binding to EGF, and are in particular the compounds generically and specifically disclosed in WO 97/02266, EP 0 564 409, WO 99/03854, EP 0520722, EP 0 566 226, EP 0 787 722, EP 0 837 063, U.S. Pat. No. 5,747,498, WO 98/10767, WO 97/30034, WO 97/49688, WO 97/38983 and, especially, WO 96/33980;
in each case where citations of patent applications or scientific publications are given in particular in the compound claims and the final products of the working examples, the subject-matter of the final products. The pharmaceutical preparations and the claims of such patent publications is hereby incorporated into the present application by reference to this publications. Comprised are likewise the corresponding stereoisomers as well as the corresponding crystal modifications, e.g. solvates and polymorphs, which are disclosed therein. The compounds used as active ingredients in the combinations disclosed herein can be prepared and administered as described in the cited documents, respectively.

In one embodiment, a preferred VEGF protein tyrosine kinase inhibitor for use in the invention is Avastin™ (bevacizumab). In a further embodiment, a preferred VEGF protein tyrosine kinase inhibitor for use in the invention is 1-(4-chloroanilino)-4-(4-pyridylmethyl)phthalazine succinate. In one embodiment, a preferred EGF protein tyrosine kinase inhibitor for use in the invention is Iressa™ (gefitinib). In a further embodiment, a preferred EGF protein tyrosine kinase inhibitor for use in the invention is Erbitux™ (cetuximab).

The structure of the active agents identified by code nos., generic or trade names may be taken from the actual edition of the standard compendium “The Merck Index” or from databases, e.g. Patents International (e.g. IMS World Publications). The corresponding content thereof is hereby incorporated by reference.

It will be understood that references to the combination partners are meant to also include the pharmaceutically acceptable salts. If the combination partners have, for example, at least one basic center, they can form acid addition salts. Corresponding acid addition salts can also be formed having, if desired, an additionally present basic center. A combination partners (a), (b), (c) or (d) having an acid group (for example COOH) can also form salts with bases. N-{5-[4-(4-methyl-piperazino-methyl)-benzoylamido]-2-methylphenyl}-4-(3-pyridyl)-2-pyrimidine-amine, i.e. combination partner (a), is preferably used in the present invention in the form of its monomesylate salt (STI571).

The combinations of the present invention can be used in the form of a combined preparation or a pharmaceutical composition.

The term “a combined preparation”, as used herein defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by, use of only any one of the combination partners. The ratio of the total amounts of the combination partner (a) and (b) to each other being administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular a synergism, e.g. a more than additive effect, additional advantageous effects, less side effects, a combined therapeutic effect in a non-effective dosage of one or both of the combination partners (a) and (b), and very preferably a strong synergism of the combination partners (a) and (b).

The term “treatment” includes the treatment of a patient in need thereof resulting in a delay of progression of the solid tumor disease. The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

A combination which comprises (a) a dual inhibitor of the EGF-R tyrosine kinase activity and the VEGF-R tyrosine kinase activity or, alternatively, a compound which decrease the activity of EGF and a compound which decreases the activity of VEGF and (b) at least one compound selected from an inhibitor of the PDGF-R tyrosine kinase activity and antineoplastic anti-metabolites, wherein the active ingredients are present in each case in free form or in the form of a pharmaceutically acceptable salt or any hydrate thereof, and optionally at least one pharmaceutically acceptable carrier; for simultaneous, separate or sequential use will be referred to hereinafter as a COMBINATION OF THE INVENTION.

It can be shown by established test models and in particular those test models described herein that a COMBINATION OF THE INVENTION is suitable for the treatment of pancreatic carcinoma. The person skilled in the pertinent art is fully enabled to select a relevant test model to prove the hereinbefore and hereinafter mentioned therapeutic indications and beneficial effects. The pharmacological activity of a COMBINATION OF THE INVENTION may, for example, be demonstrated in a clinical study or in a test procedure as essentially described hereinafter. In the studies described in the Examples, L3.6pl, a human pancreatic cancer cell, is implanted in the pancreas of nude mice. Tumor-associated endothelial cells in this model highly express phosphorylated EGFR, VEGFR, and PDGFR. Oral administration of {6-[4-(4-ethyl-piperazin-1-ylmethyl)-phenyl]-7H-pyrrolo[2,3-d]pyrimidin-4-yl}-(1-[phenyl-ethyl)-amine, a dual tyrosine kinase inhibitor against EGFR and VEGFR, decreases phosphorylation of EGFR and VEGFR. PDGFR phosphorylation is inhibited by STI571. Although intraperitoneal (i.p.) injection of gemcitabine does not inhibit tumor growth, its combination with {6-[4-(4-ethyl-piperazin-1-ylmethyl)-phenyl]-7H-pyrrolo[2,3-d]pyrimidin-4-yl}-(1-[phenyl-ethyl)-amine and STI571 produces >80% inhibition of tumor growth and prolongs survival in parallel with increases in number of tumor cells and tumor-associated endothelial cell apoptosis, decreased microvascular density, decreased proliferation rate, and prolonged survival. STI571 treatment also decreases pericyte coverage on tumor-associated endothelial cells. Thus, inhibiting phosphorylation of EGFR, VEGFR, and PDGFR in combination with gemcitabine enhances the efficacy of gemcitabine, resulting in inhibition of experimental human pancreatic cancer growth and significant prolongation of survival.

Suitable clinical studies in human patients are, for example, open label non-randomized, dose escalation studies in patients with pancreatic cancer. Such studies prove in particular superiority of the COMBINATION OF THE INVENTION over treatment with an antineoplastic anti-metabolite alone as well as the therapeutic efficacy of a COMBINATION OF THE INVENTION. The beneficial effects on pancreatic cancer can be determined directly through the results of these studies or by changes in the study design which are known as such to a person skilled in the art.

The COMBINATION OF THE INVENTION can also be applied in combination with surgical intervention, mild prolonged whole body hyperthermia and/or irradiation therapy.

It is one objective of this invention to provide a pharmaceutical composition comprising a quantity, which is jointly therapeutically effective against a solid tumor disease comprising the COMBINATION OF THE INVENTION. In this composition, the combination partners (a) and (b) can be administered together, one after the other or separately in one combined unit dosage form or in separate unit dosage forms. The unit dosage form may also be a fixed combination.

The pharmaceutical compositions for separate administration of the combination partners (a) and (b) and for the administration in a fixed combination, i.e. a single galenical compositions comprising at least two combination partners, according to the invention can be prepared in a manner known per se and are those suitable for enteral, such as oral or rectal, and parenteral administration to mammals (warm-blooded animals), including humans, comprising a therapeutically effective amount of at least one pharmacologically active combination partner alone or in combination with one or more pharmaceutically acceptable carries, especially suitable for enteral or parenteral application.

Novel pharmaceutical composition contain, for example, from about 10% to about 100%, preferably from about 20% to about 60%, of the active ingredients. Pharmaceutical preparations for the combination therapy for enteral or parenteral administration are, for example, those in unit dosage forms, such as sugar-coated tablets, tablets, capsules or suppositories, and furthermore ampoules. If not indicated otherwise, these are prepared in a manner known per se, for example by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units.

In particular, a therapeutically effective amount of each of the combination partner of the COMBINATION OF THE INVENTION may be administered simultaneously or sequentially and in any order, and the components may be administered separately or as a fixed combination. For example, the method of treatment of pancreatic cancer according to the invention may comprise (i) administration of the combination partner (a) in free or pharmaceutically acceptable salt form and (ii) administration of the combination partner (b) in free or pharmaceutically acceptable salt form, simultaneously or sequentially in any order, in jointly therapeutically effective amounts, preferably in synergistically effective amounts, e.g. in daily dosages corresponding to the amounts described herein. The individual combination partners of the COMBINATION OF THE INVENTION can be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimes of simultaneous or alternating treatment and the term “administering” is to be interpreted accordingly.

The effective dosage of each of the combination partners employed in the COMBINATION OF THE INVENTION may vary depending on the particular compound or pharmaceutical composition employed, the mode of administration, the condition being treated, the severity of the condition being treated. Thus, the dosage regimen the COMBINATION OF THE INVENTION is selected in accordance with a variety of factors including the route of administration and the renal and hepatic function of the patient. A physician, clinician or veterinarian of ordinary skill can readily determine and prescribe the effective amount of the single active ingredients required to prevent, counter or arrest the progress of the condition. Optimal precision in achieving concentration of the active ingredients within the range that yields efficacy without toxicity requires a regimen based on the kinetics of the active ingredients availability to target sites. When the combination partners employed in the COMBINATION OF THE INVENTION are applied in the form as marketed as single drugs, their dosage and mode of administration can take place in accordance with the information provided on the package insert of the respective marketed drug in order to result in the beneficial effect described herein, if not mentioned herein otherwise.

N-{5-[4-(4-methyl-piperazino-methyl)-benzoylamido]-2-methylphenyl}-4-(3-pyridyl)-2-pyrimidine-amine monomesylate, is preferably administered to a human in a dosage in the range of about 25 to 1000 mg/day, more preferably 50 to 800 mg/day and most preferably 100 to 400 mg/day. Unless stated otherwise herein, the compound is preferably administered from one to four times per day.

5-Fluorouracil may be administered to a human in a dosage range varying from about 50 to 1000 mg/m² day, e.g. 500 mg/m² day.

Capecitabine may be administered to a human in a dosage range varying from about 10 to, 1000 mg/m² day.

Gemcitabine hydrochloride may be administered to a human, in a dosage range varying from about 1000 mg/week.

Methotrexate may be administered to a human in a dosage range varying from about 5 to 500 mg/m² day.

The succinate salt described in Example 62 of WO 98/35958 may be administered to a human in a dosage range of about 50 to 1500, more preferably about 100 to 750, and most preferably 250 to 500, mg/day.

Furthermore, the present invention pertains to the use of a COMBINATION OF THE INVENTION for the treatment of pancreatic cancer and for the preparation of a medicament for the treatment of pancreatic cancer.

Moreover, the present invention provides a commercial package comprising as active ingredients COMBINATION OF THE INVENTION, together with instructions for simultaneous, separate or sequential use thereof in the treatment of pancreatic cancer.

### EXAMPLES

The following Example illustrates the invention described above, but is not, however, intended to limit the scope of the invention in any way. Other test models known assuch to the person skilled in the pertinent art can also determine the beneficial effects of the COMBINATION OF THE INVENTION.

Pancreatic Cancer Cell Line and Culture Condition. The human pancreatic cancer cell line L3.6pl is maintained in minimal essential medium supplemented with 10% fetal bovine serum (FBS), sodium pyruvate, nonessential amino acids, L-glutamine, a twofold vitamin solution (Life Technologies, Inc., Grand Island, N.Y.), and a penicillin-streptomycin mixture (Flow Laboratories, Rockville, Md.) as described previously by Hwang et al. in Clin Cancer Res 2003; 9: 6534-44.

Reagents. For oral administration, {6-[4-(4-ethyl-piperazin-1-ylmethyl)-phenyl]-7H-pyrrolo[2,3-d]pyrimidin-4-yl}-(1-[phenyl-ethyl)-amine is diluted in DMSO and STI571 is diluted in sterile water. Gemcitabine (Gemzar, Eli Lilly Co, Indianapolis, Ind.) is maintained at room temperature and dissolved in phosphate buffered saline (PBS) on the day of use. It is administered by i.p. injection.

Primary antibodies are purchased from the following manufacturers: rabbit anti-pVEGFR 2/3 (Flk-1) (Oncogene, Boston, Mass.); rabbit anti-human, mouse, rat VEGF-R(Flk-1)(C1158) (Santa Cruz Biotechnology, Santa Cruz, Calif.); rabbit anti-human pEGFR (Tyr 1173) (Biosource, Camarillo, Calif.); rabbit anti-human EGF and rabbit anti-human EGFR for paraffin samples Santa Cruz Biotechnology); rabbit anti-human EGFR for frozen samples (Zymed, San Francisco, Calif.); rabbit anti-VEGF (A20) (Santa Cruz Biotechnology); polyclonal rabbit anti-PDGFR-β, polyclonal goat anti-phosphorylated PDGFR-β, and polyclonal rabbit anti-PDGF-β (all obtained from Santa Cruz Biotechnology, Santa Cruz, Calif.); rat anti-mouse CD31 (BD PharMingen, San Diego, Calif.); mouse anti-proliferating cell nuclear antigen (PCNA) clone PC 10 (Dako NS, Copenhagen, Denmark); and rabbit anti-desmin (Dako A/S)(as a pericyte marker). The following secondary antibodies are used for colorimetric immunohistochemistry: peroxidase-conjugated goat anti-rabbit IgG; F(ab′)2 (Jackson ImmunoResearch Laboratories, Inc., West Grove, Pa.); biotinylated goat anti-rabbit (Biocare Medical, Walnut Creek, Calif.); streptavidin horseradish peroxidase (Dako NS); rat anti-mouse IgG2a horseradish peroxidase (Serotec, Harlan Bioproducts for Science, Inc., Indianapolis, Ind.); and goat anti-rat horseradish peroxidase (Jackson ImmunoResearch Laboratories, Inc.). The following fluorescent secondary antibodies are used: Alexa488 conjugated goat anti-rabbit IgG (Molecular Probes Inc., Eugene, Oreg.) and Alexa 594 conjugated goat anti-rat IgG (Molecular Probes Inc.). Terminal deoxynucleotidyl transferase-mediated nick end labeling (TUNEL) staining is performed using a commercial apoptosis detection kit (Promeg, Madison, Wis.) with modifications.

Animals and Orthotopic Implantation of Tumor Cells. Male athymic nude mice (NCI-nu) are purchased from the Animal Production Area of the National Cancer Institute Frederick Cancer Research and Development Center (Frederick, Md.). The mice are housed and maintained under specific pathogen-free conditions. The mice are used when they are 8 to 12 weeks old. To produce pancreatic tumors, L3.6pl cells are harvested from subconfluent cultures by a brief exposure to 0.25% trypsin and 0.02% EDTA. Trypsinization is stopped with medium containing 10% FBS, and the cells are washed once in serum-free medium and resuspended in Hanks' balanced salt solution (HBSS). Only suspensions consisting of single cells with greater than 90% viability are used for injection into the pancreas of nude mice as described previously (Hwang et al. in Clin Cancer Res 2003; 9: 6534-44).

Treatment of Established Human Pancreatic Carcinoma Tumors Growing in the Pancreas of Athymic Nude Mice. Twenty-one days after the intra-pancreatic injection of 0.5×10⁶ viable L3.6pl cells in 50 μl HBSS, the pancreatic tumors reach the size of 5-6-mm. At that time, the mice are randomized to the following 8 treatments (n=10): (1) Control mice: administration of water diluted at 1:20 with DMSO-0.5% Tween 80 (diluent) by oral gavage 3 times per week, daily, oral gavage with sterile water, and i.p. injections of PBS twice a week; (2) administration of diluent by oral gavage 3 times per week, daily oral gavage with sterile water, and twice weekly i.p. injections of gemcitabine (50 mg/kg); (3) oral gavage of AEE788 (50mg/kg), 3 times per week, daily oral gavage with sterile water, and twice per week i.p. injections of PBS; (4) oral gavage of AEE788 (50 mg/kg) three times per week, daily oral gavage with sterile water, and twice per week i.p. injection of gemcitabine (50 mg/kg); (5) daily oral gavage of STI571 (50 mg/kg), diluent of AEE788 by oral gavage 3 times per week and i.p. injections of PBS twice per week; (6) daily oral STI571 (50 mg/kg), oral gavage of diluent for AEE788 3 times per week, and i.p. injections of gemcitabine (50 mg/kg) twice weekly; (7) combination of oral AEE788 (50 mg/kg) 3 times per week, daily STI571 (50 mg/kg), and twice per week i.p. injections of PBS; and (8) combination of oral AEE788 (50 mg/kg) 3 times per week, STI571 (50 mg/kg) 7 times per week, and twice per week i.p. injections of gemcitabine (50 mg/kg). All mice are treated for 4 weeks and killed on day 49 of the experiment.

For survival studies, 21 days after the intra-pancreatic injection of 1.0×10⁶ tumor cells in 50 μl HBSS, at which time the tumors in the pancreas exceeded 6- to 8-mm in diameter, the mice are randomized (n=10) to one of the 8 treatment groups, as described above. The mice are killed and necropsied when they became moribund. Survival is evaluated by the Kaplan-Meier method.

Necropsy Procedures and Histological Studies. In the first treatment study, the mice are killed on day 49 after tumor cell injection, weighted, and necropsied. Tumors growing in the pancreas are excised and weighed. For immunohistochemical staining procedures, one part of the tumor tissue is fixed in formalin and embedded in paraffin and the other is embedded in OCT compound (Miles, Inc., Elkhart, Ind.), rapidly frozen in liquid nitrogen, and stored at −70° C.

Immunohistochemical (IHC) Analysis to Detect EGF, VEGF, PDGF-BB, EGFR, VEGFR, PDGFRβ, pEGFR, pVEGFR, pPDGF-R in Pancreatic Tumors. Paraffin-embedded pancreatic tumors of mice from all treatment groups are immunostained to evaluate the expression of EGF, VEGF, PDGF-BB, EGFR, VEGFR, PDGFRβ, phosphorylated (p)EGFR, pVEGFR, and pPDGFRβ. The sections are deparaffinized in xylene, dehydrated with alcohol and rehydrated in PBS. Endogenous peroxidase is blocked with 3% hydrogen peroxide in PBS. Samples are exposed to protein block (5% normal horse serum, 1% normal goat serum in PBS) and incubated overnight at 4° C. with each primary antibody at the appropriate dilution. After 1 h incubation at room temperature with peroxidase-conjugated secondary antibody, positive reaction is detected by exposure to stable 3,3′-diaminobenzidine (DAB) (Phoenix Biotechnologies, Huntsville, Ala.). Slides are counterstained with Gill's #3 hematoxylin. Sections stained for immunoperoxidase or hematoxylin and eosin are examined in a microscope equipped with a three-chip-charged coupled device (CCD) color video camera. Digital images are captured using Optimas Image Analysis software (Media Cybernetics, Md.).

IHC Determination of Proliferating Cell Nuclear Antigen (PCNA), CD31/PECAM-1 (Endothelial Cells) and TUNEL (Apoptosis). Paraffin-embedded tissues are used for IHC identification of proliferating cell nuclear antigen (PCNA). Frozen tissues used for identification of CD31/PECAM-1 are sectioned (8-10 nm), mounted on positively charged slides, and air-dried for 30 min. Frozen sections are fixed in cold acetone (5 min), in acetone/chloroform (v/v; 5 min), and again in acetone (5 min), and washed with PBS. IHC procedures are performed as described previously (Hwang et al. in Clin Cancer Res 2003; 9: 6534-44). Control samples exposed to a secondary antibody alone show no specific staining. For the quantification of mean vessel density (MVD) in sections stained for CD31, 10 random 0.159-mm² fields at X100 magnification are captured for each tumor, and microvessels are quantified. For quantification of PCNA expression, the number of positive cells is counted in 10 random 0.159-mm² fields at X100 magnification.

Analysis of apoptotic cells is performed by using a commercially available TUNEL kit (Promega) with the following modifications: Samples are fixed and incubated with an equilibration buffer followed by a reaction buffer (containing nucleotide mix and terminal deoxynucleotidyl transferase enzyme). Immunofluorescence microscopy is performed in a Zeiss Axioplan microscope (Carl Zeiss, Inc., Thornwood, N.Y.) equipped with an HBO 100 mercury lamp, narrow bandpass filters to individually select for green, red, and blue fluorescence (Chrome Technology Corp., Brattleboro, Vt.). Images are captured using a cooled CCD Hamamatsu Orca camera (Hamamatsu Corp., Bridgewater, N.J.) and Image Pro Analysis software (Media Cybernetics, Silver Spring, Md.). Photomontages are prepared using Adobe Photoshop software (Adobe Systems, Inc., San Jose, Calif.). The number of TUNEL-positive cells in 10 random 0.159-mm² fields at X100 magnification is used to quantify apoptosis.

Double Immunofluorescence Staining for CD31/PECAM-1 and EGFR, pEGFR, VEGFR, pVEGFR, PDGFRβ, pPDGFRβ, Pericytes (desmin-positive cells), and TUNEL. Frozen sections of pancreatic tumors are mounted on slides and fixed. Immunofluorescence for CD31 is performed using Alexa594 conjugated secondary antibody, and samples are again blocked briefly in a blocking solution (5% normal horse serum and 1% normal goat serum in PBS) as described above and incubated with antibody against human EGFR, pEGFR, VEGFR, pVEGFR, PDGFRβ, pPDGFRβ, or desmin at 4° C. overnight. After washes and blocking with blocking solution, samples are incubated with Alexa488 conjugated secondary antibody. Endothelial cells are identified by red fluorescence, and EGF-R, pEGFR, VEGFR, pVEGFR, PDGFRβ, pPDGFRβ and desmin positive cells (pericytes) are identified by green fluorescence. The presence of growth factor receptors and phosphorylated receptors on endothelial cells are detected by colocalization of red and green fluorescence, which appeared yellow.

The coverage of pericytes on endothelial cells is determined by counting CD31 positive cells in direct contact with desmin-positive cells and CD31-positive cells without direct association with desmin-positive cells in five randomly selected microscopic fields. TUNEL-positive apoptotic cells are detected by localized green fluorescence within cell nuclei, and endothelial cells are identified by red fluorescence. Apoptotic endothelial cells are identified by yellow fluorescence within the nuclei. Quantification of apoptotic endothelial cells is expressed as the ratio of apoptotic endothelial cells to the total number of endothelial cells in ten 0.159-mm² fields at x100 magnification.

Statistical Analysis. Body weight, tumor weight, PCNA-positive cells, mean vessel density (CD31/PECAM-1), and TUNEL-positive cells are compared using the Mann-Whitney U test. Survival analysis is computed by the Kaplan-Meier method and compared by the Log rank test.

### Results

Therapy of Human Pancreatic Cancer Growing in the Cecum of Nude Mice. In the first set of experiments, the effect of treatment with AEE788, STI571, and gemcitabine alone and in various combinations is determined against well-established (5-6 mm) pancreatic tumors. The mice are killed and necropsied on day 49 of the study (Table1). Tumor incidence in the pancreas is 100% in all treatment groups. None of the treatments significantly affects body weight. Control mice have the largest tumors (0.77 g). Treatment with STI571 or gemcitabine alone does not inhibit tumor growth, but mice treated with AEE788 have significantly smaller tumors (0.33 g: p<0.001). The combination of AEE788 and gemcitabine or AEE788 and STI571 (but not STI571 and gemcitabine) significantly decrease tumor weight in the pancreas (0.19 g, p<0.0001, 0.33 g; p<0.001 vs control, and 0.71 g, respectively). Combining AEE788, STI571, and gemcitabine for therapy produces the most significant inhibition of tumor growth (0.14 g, p<0.0001 versus control).

TABLE 1
Therapy of L3.6pl human pancreatic cancer cells implanted in the
pancreas of nude mice

| | Body weight(g) | | Tumor weight (g) | |
|---|---|---|---|---|
| Treatment | Median | (Range) | Median | (Range) |
| Control | 24.8 | (18.8-27.8) | 0.77 | (0.48-1.80) |
| Gemcitabine | 25.7 | (20.0-28.1) | 0.78 | (0.36-1.23) |
| STI571 | 23.5 | (18.7-27.2) | 0.96 | (0.45-1.83) |
| STI571 + Gemcitabine | 25.0 | (21.1-28.1) | 0.71 | (0.42-1.35) |
| AEE788 | 26.2 | (21.3-28.5) | 0.33 | (0.08-0.44)^{a} |
| AEE788 + | 25.3 | (22.1-28.8) | 0.19 | (0.05-0.40)^{b} |
| Gemcitabine | | | | |
| AEE788 + STI571 | 24.1 | (22.2-29.0) | 0.33 | (0.05-0.50)^{a} |
| AEE788 + STI571 + | 24.0 | (21.5-28.9) | 0.14 | (0.04-0.30)^{b,c} |
| Gemcitabine | | | | |

^{a}P < 0.001 vs control
^{b}P < 0.0001 vs control
^{c}P < 0.05 vs AEE788 or AEE788 and STI571.

In the next survival study, treatment begins 21 days after the intrapancreatic injection of 1.0×10⁶ L3.6 μl cells. The pancreatic tumors measure 6-8 mm in diameter. Treatment continues until the mice become moribund, at which time they are killed. Survival is analyzed using the Kaplan-Meier method. All treatments other than STI571 alone or gerncitabine alone significantly prolong survival as compared to the control treatment group. Mice treated with the combination of AEE788, STI571, and gemcitabine have the greatest prolongation of survival.

Immunohistochemical Analysis of L3.6pl Pancreatic Tumors. Tumor sections are analyzed immunohistochemically for the expression of EGF, EGFR, and pEGFR, VEGF, VEGFR, and pVEGFR and PDGF-BB, PDGFRβ and pPDGFRβ. Treatment with AEE788, STI571, gemcitabine, or any of the combination treatments do not alter the expression level of EGF, VEGF, PDGF-BB, EGFR, VEGFR, and PDGFR by the tumor cells or in the stroma cells. The phosphorylation of EGFR and VEGFR (but not PDGFR) is significantly reduced in tumors from mice treated with AEE788 alone or any combination therapy including AEE788. In contrast, PDGFRβ (but not EGFR or VEGFR) phosphorylation is inhibited in tumors from mice treated with STI571 alone or combination therapy including STI571. These data confirm that at the concentration administered to mice, the PTK inhibitors produces specific inhibition of their respective target receptors. The combination therapies with AEE788 and STI571 and with AEE788, STI571, and gemcitabine inhibits phosphorylation of all three receptors.

EGF-R, VEGFR, PDGFR, pEGFR, pVEGFR and pPDGFR on Tumor-associated Endothelial Cells. To determine whether tumor-associated endothelial cells express EGFR, VEGFR, PDGFRβ, pEGFR, pVEGFR, or pPDGFRβ a double immunofluorescence staining technique is used. Tumor-associated endothelial cells from all treatment groups express similar levels of EGFR, VEGFR, and PDGFRβ. The phosphorylation of EGFR and VEGFR is diminished on endothelial cells from tumors of mice treated with AEE788 or combination treatments including AEE788.

Phosphorylation of the PDGFRβis decreased on endothelial cells from tumors of mice treated with STI571 or combination treatments including STI571. Administration of AEE788 and STI571 or AEE788, STI571, and gemcitabine inhibited phosphorylation of EGFR, VEGFR, and PDGFRβ on tumor-associated endothelial cells.

Cell Proliferation (PCNA), Apoptosis (TUNEL), and Mean Vessel Density (MVD). Cell proliferation is evaluated by staining for PCNA. In tumors from control mice, the median number of PCNA-positive cells is 371±88. As shown in Table 2, treatment with gemcitabine alone or STI571 alone decreases the number of dividing PCNA-positive cells. A significant decrease of PCNA-positive cells is found in tumors from all other treatment groups, with the highest inhibition produced in tumors from mice treated with AEE788, STI571, and gemcitabine (155±54, P<0.001).

TABLE 2
Immunohistochemical analysis of L3.6pl human pancreatic
cancer cells growing in the pancreas of nude mice

| | Tumor cells | | Endothelial cells | |
|---|---|---|---|---|
| Treatment | PCNA^{a,c} | TUNEL^{b,c} | CD31^{c} | TUNEL⁺ (%)^{c} |
| Control | 371 ± 88 | 1 ± 1 | 46 ± 11 | 0 ± 0 |
| Gemcitabine | 305 ± 71 | 8 ± 3^{f} | 38 ± 7 | 1 ± 1 |
| STI571 | 301 ± 49 | 6 ± 2 | 37 ± 7 | 0 ± 0 |
| STI571 + | 254 ± 48^{e} | 11 ± 4^{f} | 34 ± 8^{d} | 0 ± 1 |
| Gemcitabine | | | | |
| AEE788 | 233 ± 54^{e} | 14 ± 4^{f} | 25 ± 5^{d} | 3 ± 3^{e} |
| AEE788 + | 187 ± 48^{f} | 22 ± 7^{f,k} | 28 ± 7^{f,k} | 8 ± 6^{e} |
| Gemcitabine | | | | |
| AEE788 + | 204 ± 69^{e} | 18 ± 6^{f} | 21 ± 5^{f} | 5 ± 5^{e} |
| STI571 | | | | |
| AEE788 + | 155 ± 54^{f,h} | 30 ± 10^{g,i,j} | 1.6 ± 6^{f,l} | 8 ± 5^{e} |
| STI571 + | | | | |
| Gemcitabine | | | | |

^{a}PCNA, proliferating cellular nuclear antigen;
^{b}TUNEL, terminal deoxynucleotidyltransferase-mediated nick end labeling;
^{c}Median ± S.D.;
^{d}P < 0.05 vs control;
^{e}P < 0.01 vs control;
^{f}P < 0.001 vs control;
^{g}P < 0.001 vs control;
^{h}P < 0.05 vs AEE788;
ⁱP < 0.01 vs AEE788;
^{j}P < 0.05 vs AEE788 + STI571;
^{k}P < 0.05 vs AEE788.

The induction of apoptosis in the pancreatic tumors is evaluated by the TUNEL method (Table 2). In tumors from control-treated mice, the median number of apoptotic tumor cells is minimal (1±1). The number of apoptotic cells in tumors from mice in all other treatment groups (except those treated with only STI571) increases, with the highest produced by therapy with the combination of AEE788, STI571, and gemcitabine (30±10).

MVD in the tumors is determined by IHC staining with antibodies against CD31 (Table 2). The median number of CD31-positive tumor cells from control mice is 46±11. Treatment with gemcitabine alone or STI571 alone does not decrease MVD. The number of CD31-positive cells is significantly decreased in tumors from all other treatment groups, with the largest decrease in MVD in tumors from mice treated with AEE 788, STI571, and gemcitabine (16±6) (P<0.001).

Immunofluorescence Double Staining for CD31/PECAM-1 and TUNEL It is determined whether therapy is associated with apoptosis of endothelial cells by using the CD31/TUNEL fluorescent double-labeling technique. Tumors from control mice have no apoptosis in tumor-associated endothelial cells. Treatment of mice with AEE788, STI571, and gemcitabine produce a median of 8±5% apoptosis in tumor-associated endothelial cells (Table 2).

Further details of the study are described by Kenji Yokoi, Takamitsu Sasaki, Corazon D. Bucana, Dominic Fan, Cheryl H. Baker, Yasuhiko Kitadai, Toshio Kuwai, James L. Abbruzzese, and Isaiah J. Fidler in Cancer Research, 2005 Nov. 15; 65(22):10371-80, which publication is incorporated into the present specification by reference.

1. (canceled)

2. A method of treating a warm-blooded animal having pancreatic cancer comprising administering to the warm-blooded animal a dual inhibitor of the EGF-R tyrosine kinase activity and the VEGF-R tyrosine kinase activity in a quantity which is therapeutically effective against pancreatic cancer in which the dual inhibitor of the EGF-R tyrosine kinase activity and the VEGF-R tyrosine kinase activity can also be present in the form of a pharmaceutically acceptable salt.

3-5. (canceled)

6. A method of treating a warm-blooded animal having pancreatic cancer comprising administering to the warm-blooded animal a combination comprising (a) an inhibitor of the EGF-R tyrosine kinase activity and (b) an inhibitor of the VEGF-R tyrosine kinase activity in a quantity which is therapeutically effective against pancreatic cancer in which the components of the combination can also be present in the form of their pharmaceutically acceptable salts.

7. A combination comprising (a) a dual inhibitor of the EGF-R tyrosine kinase activity and the VEGF-R tyrosine kinase activity or, alternatively, a compound which decrease the activity of EGF and a compound which decreases the activity of VEGF and (b) at least one compound selected from an inhibitor of the PDGF-R tyrosine kinase activity and antineoplastic antimetabolites, wherein the active ingredients are present in each case in free form or in the form of a pharmaceutically acceptable salt or any hydrate thereof, and optionally at least one pharmaceutically acceptable carrier; for simultaneous, separate or sequential use.

8. Combination according to claim 7 wherein compound (b) is selected from bevacizumab and 1-(4-chloroanilino)-4-(4-pyridylmethyl)phthalazine succinate.

9. Combination according to claim 7 wherein compound (a) is selected from gefitinib and cetuximab.

10. The combination according to claim 7 comprising wherein compound (a) is a 7H-pyrrolo[2,3-d]pyrimidine derivatives of formula I 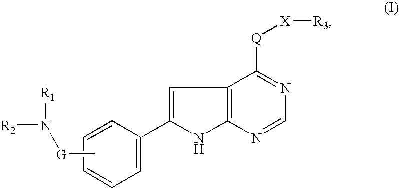
wherein
R₁ and R₂ are each independently of the other hydrogen, unsubstituted or substituted alkyl or cycloalkyl, a heterocyclic radical bonded via a ring carbon atom, or a radical of the formula R₄—Y—(CαZ)- wherein R₄ is unsubstituted, mono- or disubstituted amino or a heterocyclic radical, Y is either not present or lower alkyl and Z is oxygen, sulfur or imino, with the proviso that R₁ and R₂ are not both hydrogen; or
R₁ and R₂ together with the nitrogen atom to which they are attached form a heterocyclic radical;
R₃ is a heterocyclic radical or an unsubstituted or substituted aromatic radical;
G is C₁-C₇-alkylene, —C(═O)—, or C₁-C₆-alkylene-C(═O)— wherein the carbonyl group is attached to the NR₁R₂ moiety;
Q is —NH— or —O—, with the proviso that Q is —O— if G is —C(═O)— or C₁-C₆-alkylene-C(═O)—; and
X is either not present or C₁-C₇-alkylene, with the proviso that a heterocyclic radical R₃ is bonded via a ring carbon atom if X is not present;
or a salt of the said compounds,
and (b) at least one compound selected from N-phenyl-2-pyrimidine-amine derivatives, 5-fluorouracil, capecitabine, gemcitabine, methotrexate and edatrexate.

11. The combination according to claim 7 comprising (a) {6-[4-(4-ethyl-piperazin-1-ylmethyl)-phenyl]-7H-pyrrolo[2,3-d]pyrimidin-4-yl}-(1-phenyl-ethyl)-amine and (b) at least one compound selected from N-{5-[4-(4-methyl-piperazino-methyl)-benzoylamido]-2-methylphenyl}-4-(3-pyridyl)-2-pyrimidine-amine and gemcitabine.

12. The combination according to claim 11 wherein N-{5-[4-(4-methyl-piperazino-methyl)-benzoylamido]-2-methylphenyl}-4-(3-pyridyl)-2-pyrimidine-amine is used in the form of its mono-methanesulfonate salt.

13. (canceled)

14. A method of treating a warm-blooded animal having pancreatic cancer comprising administering to the warm-blooded animal a combination according to claim 7 in a quantity which is therapeutically effective against pancreatic cancer in which the components of the combination can also be present in the form of their pharmaceutically acceptable salts.

15. A commercial package comprising (a) a dual inhibitor of the EGF-R tyrosine kinase activity and the VEGF-R tyrosine kinase activity or, alternatively, a compound which decrease the activity of EGF and a compound which decreases the activity of VEGF and (b) at least one compound selected from an inhibitor of the PDGF-R tyrosine kinase activity and antineoplastic anti-metabolites, wherein the active ingredients are present in each case in free form or in the form of a pharmaceutically acceptable salt or any hydrate thereof, and optionally at least one pharmaceutically acceptable carrier; together with instructions for the simultaneous, separate or sequential use thereof in the treatment of pancreatic cancer.
